(19) United States
(12) Patent Application Publication
(10) Pub. No.: US 2025/0152780 A1
(43) Pub. Date: May 15, 2025

(54) METHOD FOR PREPARING BIOLOGICAL VALVE MATERIAL BY MEANS OF DOUBLE-BOND POLYMERIZATION AFTER ALDEHYDE CROSSLINKING, AS WELL AS BIOLOGICAL VALVE MATERIAL AND USE

(71) Applicant: SICHUAN UNIVERISTY, Chengdu (CN)

(72) Inventors: Yunbing Wang, Chengdu (CN); Cheng Zheng, Chengdu (CN); Li Yang, Chengdu (CN); Gaocan Li, Chengdu (CN); Rifang Luo, Chengdu (CN); Dajun Kuang, Chengdu (CN); Linhe Jin, Chengdu (CN); Caili Ma, Chengdu (CN)

(21) Appl. No.: 19/026,783

(22) Filed: Jan. 17, 2025

Related U.S. Application Data
(63) Continuation of application No. PCT/CN2022/132870, filed on Nov. 18, 2022.

(30) Foreign Application Priority Data
Nov. 15, 2022 (CN) 202211431077.5

(51) Int. Cl. A61L 27/36; A61F 2/24; A61L 27/16; A61L 27/50

(52) U.S. Cl. CPC A61L 27/3687; A61F 2/2415; A61F 2/2418; A61L 27/16; A61L 27/3625; A61L 27/507; A61L 2430/20; A61L 2430/40

(57) ABSTRACT

Disclosed is a method for preparing biological valve material by means of double-bond polymerization after aldehyde crosslinking, as well as the biological valve material and use therefor. The preparation method includes: step S110, contacting the biomaterial with an aldehyde-based crosslinking agent solution for crosslinking; Step S120: soaking the biomaterial processed in Step S110 in a solution containing a first functional monomer for reaction to introduce first carbon-carbon double bonds; wherein the first functional monomer has first carbon-carbon double bonds and an epoxyethane group; and Step S200, performing polymerization of the carbon-carbon double bonds under an action of an initiator to obtain the biological valve material. In this disclosure, double bonds are introduced into the glutaraldehyde crosslinked biological valve material, and polymerization of the double bonds is further initiated, improving the stability of the glutaraldehyde crosslinked material and further reducing the risk of calcification caused by structural degradation.

### CROSS-REFERENCE TO RELATED APPLICATIONS

The present application is a Continuation Application of PCT Application No. PCT/CN2022/132870, filed on Nov. 18, 2022, which claims the priority of Chinese Patent Application No. 202211431077.5, filed on Nov. 15, 2022, the entire contents of which are hereby incorporated by reference.

### TECHNICAL FIELD

The present disclosure relates to the technical field of interventional materials, and in particular to a method for preparing biological valve material by means of double-bond polymerization after aldehyde crosslinking, as well as biological valve material and use.

### BACKGROUND

Biological heart valves are generally prepared from the porcine or bovine pericardium, and are used to replace human heart valves with functional defects. Compared to mechanical heart valves, biological heart valves have many advantages: patients do not need to take anticoagulants for a long time after implantation of biological heart valves, and biological heart valves can be implanted using minimally invasive surgical methods. These advantages have gradually made biological heart valves the mainstream of the market in clinical applications.

Almost all biological valve products on the market are prepared by crosslinking with glutaraldehyde. Glutaraldehyde can improve the mechanical properties of the pericardium and reduce its immunogenicity to a certain extent. However, the stability and crosslinking degree of glutaraldehyde crosslinked biological valves are still low, which will lead to the degradation of components after implantation, causing the structure to be damaged and structurally degraded. On the other hand, the degradation of biological valve components will further induce mechanical damage and calcification, affecting the normal function of the valve and reducing its service life.

Glutaraldehyde crosslinking is still the mainstream method for current biological valve products. Therefore, further modification of biological valves based on glutaraldehyde crosslinking to improve their crosslinking degree and stability is of great significance to scientific research and the development of related industries.

The applicant of the present disclosure has long been committed to the research of biological heart valves. For example, in the previous research, the Chinese patent publication No. CN 114748694A disclosed a co-crosslinked biological valve material, preparation method therefor and use thereof, wherein during the crosslinking treatment, the biological valve material was functionally modified by introducing functional monomers for co-crosslinking. Chinese patent publication Nos. CN 114748693A, CN 114748697A, CN 114748696A and CN 114748695A disclosed the biological valve preparation methods, wherein when the functional monomers were added for co-crosslinking, carbon-carbon double bonds were introduced from the functional monomers as a further crosslinking basis, so that the modification of the biological valve material was completed through two rounds of crosslinking.

In the aforementioned research, whether it is introducing functional monomers for co-crosslinking modification through glutaraldehyde crosslinking, or introducing carbon-carbon double bonds as the basis for further crosslinking during the co-crosslinking process, new modified substances are introduced during the glutaraldehyde crosslinking process to participate in the crosslinking reaction.

### SUMMARY

The present disclosure provides a method for preparing a biological valve material by means of double-bond polymerization after aldehyde crosslinking, a biological valve material and use therefor. After glutaraldehyde crosslinking, functional monomers with carbon-carbon double bonds are introduced from the active groups on the glutaraldehyde crosslinked valve, such as residual amino groups, hydroxyl groups, carboxyl groups, etc., providing the glutaraldehyde crosslinked valve with a controllable chance and range of crosslinking.

A method for preparing biological valve material by means of double-bond polymerization after aldehyde crosslinking, including:

Step S110: contacting a biomaterial with an aldehyde-based crosslinking agent solution for crosslinking;

Step S120: soaking the biomaterial treated in step S110 in a solution containing a first functional monomer for a chemical reaction to introduce the first carbon-carbon double bonds; the first functional monomer has a first carbon-carbon double bonds and an ethylene oxide group; and

Step S200: performing polymerization of carbon-carbon double bonds under an action of an initiator to obtain the biological valve material.

Optionally, the aldehyde-based crosslinking agent is glutaraldehyde or formaldehyde.

Optionally, the biomaterial is animal tissue, including one or more of the following: pericardium, valve, intestinal valve, meninges, lung valve, blood vessel, skin or ligament.

Optionally, the animal tissue is a fresh animal tissue or a decellularized biological tissue.

The step S200 includes adding an initiator to the system treated in the previous step; or washing the biomaterial treated in the previous step and then soaking it in a solution containing the initiator.

Optionally, the initiator is a single initiator or a mixed initiator.

Optionally, the mixed initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or potassium persulfate and tetramethylethylenediamine, or ammonium persulfate and tetramethylethylenediamine, or sodium persulfate and tetramethylethylenediamine; and the concentration of each component in the mixture is in the range of 1 to 100 mM.

Optionally, the single initiator is any component of each mixed initiator.

Optionally, in step S200, the double-bond polymerization time is in the range of 3 to 24 h.

Optionally, the first functional monomer is selected from at least one of the following: allyl glycidyl ether, glycidyl methacrylate and glycidyl acrylate.

Optionally, in step S110, the w/w concentration of the aldehyde-based crosslinking agent solution is in the range of 0.1% to 5%, and the crosslinking time is in the range of 0.5 h to 120 h.

Optionally, in step S120, the w/w concentration of the first functional monomer in the solution containing the first functional monomer is in the range of 1% to 10%; and the reaction time is in the range of 2 h to 120 h.

Optionally, the solution containing the first functional monomer only includes the first functional monomer and a solvent that does not participate in the chemical reaction.

Optionally, the solvent in the solution containing the first functional monomer is one or more of the following: water, physiological saline, and a neutral pH buffer, and an aqueous solution of any one of methanol, ethanol, ethylene glycol, propanol, 1,2-propylene glycol, 1,3-propylene glycol, isopropanol, butanol, isobutanol, 1,2-butylene glycol, 1,3-butylene glycol, 1,4-butylene glycol, and glycerol.

The present disclosure further provides a biological valve material prepared by the preparation method, the method includes:

Step S110: contacting the biomaterial with an aldehyde-based crosslinking agent solution for crosslinking;

Step S120: soaking the biomaterial treated in step S110 in a solution containing a first functional monomer for a chemical reaction to introduce the first carbon-carbon double bonds; wherein the first functional monomer has a first carbon-carbon double bonds and an ethylene oxide group; and

Step S200: performing polymerization of carbon-carbon double bonds under an action of an initiator to obtain the biological valve material.

The present disclosure further provides a biological valve, including a stent and leaflets, wherein the leaflets are made of the biological valve material.

Optionally, the biological valve is a prosthetic heart valve.

The present disclosure further provides an interventional system, including a prosthetic heart valve and a catheter assembly, wherein the prosthetic heart valve is delivered by the catheter assembly after being folded. The prosthetic heart valve includes a stent and leaflets, and the leaflets are made of the biological valve material.

Compared with the prior art, the present invention has at least one of the following benefits:

(1) Based on the biological valve material crosslinked by glutaraldehyde, in the present disclosure, double bonds are introduced to the glutaraldehyde crosslinked biological valve material through double-bond modification as the basis for secondary crosslinking, and then a polymerization of the double bonds on the glutaraldehyde crosslinked biological valve material is initiated for secondary crosslinking, which can further increase the crosslinking degree of the biological valve material, thereby improving the stability of the biological valve material.

(2) In the present disclosure, the stability of glutaraldehyde crosslinked biological valve material is improved and the risk of calcification caused by structural degradation is reduced by introducing double bonds onto the biological valve material crosslinked with glutaraldehyde and then triggering the polymerization of double bonds. Thus, the material also has certain anti-calcification properties.

(3) Compared with the applicant's previous research on methods for modifying biological valve materials that introduced carbon-carbon double bonds by adding functional monomers for co-crosslinking during glutaraldehyde modification, in the process of modifying the biological valve materials of the present disclosure, the glutaraldehyde crosslinking treatment is firstly performed and then the residual amino groups and active groups such as hydroxyl and carboxyl groups on the glutaraldehyde crosslinked valve are chemically conjugated to the functional monomers having carbon-carbon double bonds. The functional monomers with carbon-carbon double bonds undergo chemical reactions with the amino groups, hydroxyl groups and carboxyl groups on the glutaraldehyde crosslinked valve through the ethylene oxide groups, thus the carbon-carbon double bonds are mainly grafted on the surface of the biological valve material. As there is no other substance that can participate in the crosslinking reaction during the process of glutaraldehyde crosslinking modification of the biological valve material, the original fiber structure of the biomaterial can be better protected. This can effectively ensure the mechanical properties of the valve while maintaining the orientation direction of the original fibers of the biomaterial, thereby avoiding the problem of directly adding double-bond functional monomers during crosslinking in previous research, which may damage the original fiber orientation of the biomaterial and increase fiber disorder.

### BRIEF DESCRIPTION OF DRAWINGS

FIG. 1 is a process flow diagram of a preferred embodiment of the present disclosure;

FIG. 2 is a reaction schematic diagram of a preferred embodiment of the present disclosure;

FIG. 3 is an image showing the alizarin red staining result of control group 1 (glutaraldehyde crosslinked porcine pericardium) after subcutaneous implantation in rat for 30 days;

FIG. 4 is an image showing the alizarin red staining result of sample 1 of Example 1 after subcutaneous implantation in rat for 30 days;

FIG. 5 is an image showing the alizarin red staining result of sample 5 of Example 5 after subcutaneous implantation in rat for 30 days;

FIG. 6 is an image showing the alizarin red staining result of sample 7 of Example 7 after subcutaneous implantation in rat for 30 days;

FIG. 7 is a schematic structural view of a prosthetic heart valve of the present disclosure;

FIG. 8 is a schematic structural view of an intervention system of the present disclosure.

### DESCRIPTION OF EMBODIMENTS

The technical solutions according to the embodiments of the present disclosure will be described clearly and fully in combination with the drawings according to the embodiments of the present disclosure. Apparently, the described embodiments are not all embodiments of the present disclosure, but only part of the embodiments of the present disclosure. Based on the disclosed embodiments, all other embodiments obtained by those skilled in the art without creative work fall into the scope of this invention.

Unless otherwise defined, all technical and scientific terms used herein have the same meaning as commonly understood by a person skilled in the art. The terms in the description of the present disclosure are used to describe specific embodiments, and not to limit the present disclosure.

Almost all existing biological valve products on the market are prepared by crosslinking with glutaraldehyde. Glutaraldehyde can improve the mechanical properties of the pericardium and reduce its immunogenicity to a certain extent, but glutaraldehyde crosslinked biological valves still have problems of low stability and crosslinking degree. This may lead to the degradation of the components after implantation, causing structural damage and consequently structural degradation. Furthermore, the degradation of biological valve components will further induce mechanical damage and calcification, affecting the normal function of the valve and reducing its service life. Glutaraldehyde crosslinking is still the mainstream method for current biological valve products. Therefore, on the basis of glutaraldehyde crosslinking, further crosslinking and modifying the biological valves to improve their crosslinking degree and stability is of great significance for the development of scientific research and related industries.

In the present disclosure, the double bonds are further introduced and initiated to crosslink on the basis of glutaraldehyde crosslinking, that is, on the basis of the glutaraldehyde crosslinked valve, the first functional monomer (containing the first carbon-carbon double bonds and the epoxy ethane group) is chemically bonded with the glutaraldehyde crosslinked biological valve to introduce the first carbon-carbon double bonds, which will improve the crosslinking degree, stability, mechanical properties and anti-calcification of glutaraldehyde crosslinked biological valve material

In one embodiment, the method specifically includes (see FIG. 1):

S110: soaking the biological valve material in an aldehyde-based crosslinking agent solution for crosslinking to prepare a glutaraldehyde crosslinked biological valve material;

S120: soaking the glutaraldehyde crosslinked biological valve material prepared in step S110 in a solution containing a double-bond reagent (the first functional monomer) for double-bond modification to prepare a double-bond biological valve material; wherein the double-bond reagent (the first functional monomer) has at least one first carbon-carbon double bonds and an ethylene oxide group, and

S200: contacting the biological valve material treated in step S120 with an initiator to initiate double-bond polymerization.

In the present disclosure, the biomaterial firstly undergoes a crosslinking reaction with an aldehyde-based crosslinking agent (S110) and then reacts with the active group of the first functional monomer to graft the first carbon-carbon double bonds (S120). During the preparation process, the aldehyde-based crosslinking agent is firstly added to react with part of the amino groups of the biomaterial, and then the first functional monomer is added so that the first carbon-carbon double bonds are directly introduced through the reaction of the remaining amino groups and other groups (such as hydroxyl and carboxyl groups) on the biomaterial with the active group of the first functional monomer. In this scheme, the active group of the first functional monomer is an oxirane group. In addition to the remaining amino groups on the biomaterial participating in the reaction, the hydroxyl and carboxyl groups can also react with the oxirane group and thus participate in the chemical reaction. The first carbon-carbon double bonds introduced through the chemical reaction undergo polymerization under the action of the initiator to further form a crosslinked network, improving the anti-coagulation, anti-calcification, elasticity and other properties of the glutaraldehyde crosslinked biological valve.

The reaction principle of this disclosure is explained as follows:

In this double-bond crosslinking scheme, after the biological valve material is crosslinked with glutaraldehyde, the solution containing the first functional monomer (i.e., the double-bond reagent) is further used to introduce the first carbon-carbon double bonds for double-bonding the glutaraldehyde crosslinked biological valve material, wherein the first functional monomer (i.e., the double-bond reagent) has both the first carbon-carbon double bonds and the oxirane group.

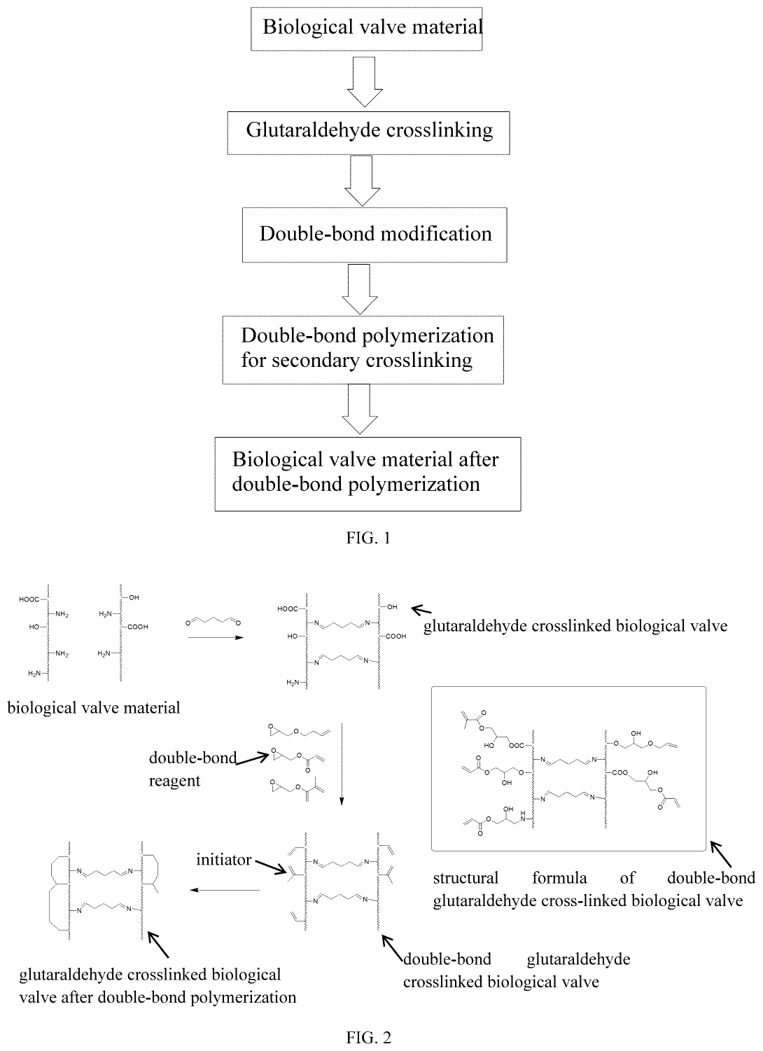

For case of understanding of the chemical mechanism involved in this scheme, FIG. 2 is illustrated as an example for explanation. The first functional monomer (i.e., the double-bond reagent) is used to modify the glutaraldehyde crosslinked biological valve material. A ring-opening reaction occurs between the oxirane group of the first functional monomer (i.e., the double-bond reagent) and the hydroxyl groups, carboxyl groups as well as the remaining small amount of amino groups after glutaraldehyde crosslinking on the glutaraldehyde crosslinked biological valve material, thereby directly introducing first carbon-carbon double bonds to the glutaraldehyde crosslinked biological valve material. Further, a polymerization of these double bonds on the glutaraldehyde crosslinked biological valve material is initiated to achieve secondary crosslinking and complete the post-crosslinking treatment of the biological valve material. The crosslinking degree of the biological valve material after secondary crosslinking will be further improved, as well as its stability, mechanical properties and anti-calcification properties.

The carbon-carbon double bonds are introduced after crosslinking with glutaraldehyde, which mainly grafted on the surface of the biological valve material. Since there are no other substances that can participate in the crosslinking reaction added in the process of glutaraldehyde crosslinking modification of the biological valve material, the original fiber structure of the biomaterial can be better protected. This can maintain the original orientation direction of the fibers of the biomaterial while effectively ensuring the mechanical properties of the valve, avoiding the problem of directly adding double-bond functional monomers during crosslinking in previous research, which may damage the original fiber orientation of the biological material and increase fiber disorder.

Optionally, in step S120 of the present disclosure, the first carbon-carbon double bonds are introduced through a non-condensation chemical bonding.

Optionally, in step S110, the biomaterial does not undergo any chemical reaction involving other reagents before being treated with the aldehyde-based crosslinking agent.

Further optionally, in the reaction system of step S120, the first carbon-carbon double bonds are provided by the first functional monomer having the active group, and the reaction raw materials in step S100 only include the biomaterial, the first functional monomer and the aldehyde-based crosslinking agent.

In step S110:

The crosslinking agent in the present disclosure is an aldehyde-based crosslinking agent used in current mainstream crosslinking methods. Optionally, the aldehyde-based crosslinking agent is selected from glutaraldehyde and formaldehyde.

Optionally, the concentration of the glutaraldehyde solution is in the range of 0.1% to 5% (w/w), and the crosslinking time may be any time in the range of 0.5 h to 120 h.

The biomaterials used here are conventional biomaterials used in the existing glutaraldehyde crosslinking processes with collagen content ranging from 60% to 90%. Further, the biomaterials are animal tissues originating from the pig, cattle, horse or sheep, including one or more of the following: pericardium, valve, intestinal valve, meninges, lung valve, blood vessel, skin or ligament.

Optionally, the animal tissue is fresh animal tissue or decellularized biological tissue.

Optionally, in the decellularization, the biological tissue is treated with a surfactant as follows:
the biological tissue is decellularized with an ionic surfactant; or
the biological tissue is decellularized with a non-ionic surfactant.

The ionic surfactant is mainly used to lyse cells, and the non-ionic surfactant is mainly used to remove lipid substances (such as phospholipids).

Optionally, the ionic surfactant is at least one of the following: sodium deoxycholate, fatty acid potassium soap, sodium dodecyl sulfate, sodium cholate, hexadecyltrimethylammonium bromide, fatty acid potassium salt, and alkyldimethylsulfonpropyl betaine.

Optionally, the nonionic surfactant is at least one of Triton and Tween.

In step S120:

Optionally, the double-bond reagent, i.e., the first functional monomer, is selected from at least one of allyl glycidyl ether, glycidyl methacrylate and glycidyl acrylate.

Optionally, the concentration of the double-bond reagent in the solution containing the first functional monomer (i.e., the double-bond reagent) is in the range of 1% to 10% (w/w), and the reaction time for the double-bond modification is in the range of 2 h to 120 h.

Optionally, the solvent in the solution containing the first functional monomer, i.e., the double-bond reagent, is one or more of the following: water, physiological saline, neutral pH buffer, or an aqueous solution of methanol, ethanol, ethylene glycol, propanol, 1,2-propylene glycol, 1,3-propylene glycol, isopropanol, butanol, isobutanol, 1,2-butylene glycol, 1,3-butylene glycol, 1,4-butylene glycol, and glycerol.

Optionally, the biofilm material treated in S110 is taken out and washed or directly placed in the solution containing a double-bond reagent (first functional monomer).

In step S200:

the biological valve material treated in step S120 is cleaned with deionized water and then immersed in the initiator solution, or the initiator is directly added to the reaction system in step S120 to initiate the polymerization reaction; wherein the latter is commonly known as the one-pot method;

Optionally, the solvent in the solution containing the initiator is water, physiological saline or neutral pH buffer.

Regarding the concentration of the initiator as mentioned above, in the one-pot method, it may be understood as the concentration of the solution of the reaction system in step S120 in the one pot method, while in the step-by-step method, it may be understood as the concentration of the solution containing the initiator.

Optionally, the initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or potassium persulfate and tetramethylethylenediamine, or ammonium persulfate and tetramethylethylenediamine, or sodium persulfate and tetramethylethylenediamine; and the concentration of each component in the mixture is in the range of 1 to 100 mM.
optionally, the double-bond polymerization time is preferably in the range of 3 to 24 h.

In the present disclosure, all reactions in S110, S120 and S200 can be carried out at 0 to 50° C. unless otherwise specified. Preferably, the temperature does not need to be specially controlled and can be at room temperature. It is suitable not to exceed the human body's adaptive temperature, preferably in the range of 36 to 37° C.

In the present disclosure, all reactions in S110, S120 and S200 may be either static reactions or dynamic reactions unless otherwise specified. Dynamic reactions may be conducted using equipment capable of circulating the solution, such as a peristaltic pump, or through shaking at a rotation speed ranging from 10 rpm to 150 rpm. The peristaltic cycle or shaking may be carried out continuously or intermittently.

In the present disclosure, optionally, dehydration and drying treatment after the double-bond polymerization is completed are carried out to prepare a dry valve. Further, after the double-bond polymerization is completed, the biological valve material is conventionally cleaned, softened, and then dehydrated and dried.

The cleaning solution may be one or more of water, physiological saline, ethanol, isopropanol or neutral pH buffer solution. The pH may be adjusted to be in the range of 5.0 to 9.5 before and during use, or there is no need to adjust the pH.

Optionally, the dehydration treatment involves exposing the valve piece subjected to double-bond polymerization or the valve formed by sewing valve pieces to a dehydration solution.

Optionally, the dehydration solution is a mixed solution of alcohol solution and water with the alcohol solution accounting for 20% to 90% (v/v). The alcohol reagent may be one or both of ethanol and isopropanol.

Optionally, the drying treatment involves exposing the dehydrated valve piece or valve to a softener solution for 20 minutes to 10 h.

Optionally, the main component of the softener solution is one or a mixed solution of glycerol and polyethylene glycol, with the glycerol concentration ranging from 10% to 100% (v/v), and the remaining component is one or more of water, ethanol, isopropanol, etc., accounting for 0 to 90% (v/v).

Optionally, the valve sterilization method after drying treatment may be one of ethylene oxide sterilization or electron beam sterilization.

The biological valve material prepared by the above methods may be used for interventional biological valves, for example, through minimally invasive intervention; or the biological valve material prepared by the above methods may be used for surgical biological valves, for example, through surgical implantation.

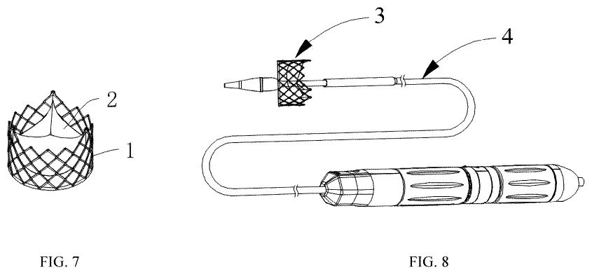

As shown in FIG. 7, a prosthetic heart valve according to an embodiment includes a stent 1 and leaflets 2 connected in the stent 1. The stent is generally cylindrical, and the side wall thereof has a hollow mesh structure. There is a blood flow channel defined in the stent, and the leaflets cooperate with each other to control the opening and closing of the blood flow channel in the stent.

Depending on the release modes, the stent is made of corresponding materials during processing, such as nickel-titanium alloy that has shape memory and can self-expand in the human body, or stainless steel that is released by ball expansion, etc. The stent itself may be formed by cutting pipes or braiding wires. The leaflets may be connected to the stent by sewing, bonding or integral molding.

For positioning in the human body, positioning structures that can interact with surrounding native tissues may be provided on the periphery of the stent, such as anchors, arms, etc. In order to prevent peripheral leakage, skirts or anti-peripheral leakage materials or the like may be provided on the inside and/or outside of the stent. The leaflets, skirts or anti-peripheral leakage materials may all be made of the biological valve materials in the above embodiments.

As shown in FIG. 8, in case of intervention through catheter, the prosthetic heart valve 3 and a corresponding delivery system form a valve intervention system. The delivery system includes a catheter assembly 4 and a handle for controlling the catheter assembly. The prosthetic heart valve is in a radially compressed state during delivery in the human body, and is radially expanded in the human body after releasing the restriction of the catheter assembly or being expanded by ball expansion.

Specific examples are illustrated in the following.

### Control Group 1

Freshly collected porcine pericardium was washed with distilled water at 4° C. and 100 RPM for 2 h, then immersed in 0.30% (w/w) glutaraldehyde solution and crosslinked at room temperature and 100 RPM for 48 h to obtain control sample 1.

### Example 1

In this embodiment, freshly collected porcine pericardium is washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water, and then soaked in a propanol aqueous solution of 5% (v/v) glycidyl methacrylate at room temperature for double-bond modification of the glutaraldehyde crosslinked porcine pericardium for 72 h, and the solvent of the double-bond solution was 20% (v/v) propanol aqueous solution.

After the double-bond modification was completed, the double-bond glutaraldehyde crosslinked porcine pericardium was washed with deionized water; and then soaked in a mixture of 20 mM potassium persulfate and 10 mM sodium bisulfite to initiate polymerization of the double bonds on the double-bond glutaraldehyde crosslinked porcine pericardium and react at 37° C. for 8 h, thereby obtaining the porcine pericardium after double-bond polymerization, which was recorded as sample 1.

### Example 2

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an isopropanol aqueous solution of 6% (v/v) glycidyl acrylate at room temperature for double-bond modification of the glutaraldehyde crosslinked porcine pericardium for 72 h, and the solvent of the double-bond solution was 20% (v/v) isopropanol aqueous solution.

After the double-bond modification was completed, the double-bond glutaraldehyde crosslinked porcine pericardium was washed with deionized water; and then soaked in a mixture of 20 mM ammonium persulfate and 5 mM sodium bisulfite to initiate the polymerization of the double bonds on the double-bond glutaraldehyde crosslinked porcine pericardium and react at 37° C. for 8 h, thereby obtaining the porcine pericardium after double-bond polymerization, which was recorded as sample 2.

### Example 3

Freshly collected porcine pericardium was washed with distilled water at 4° C. and 100 RPM for 2 h, soaked in 0.30% (w/w) glutaraldehyde solution at room temperature at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an isopropanol aqueous solution of 4% (v/v) glycidyl acrylate and 4% (v/v) allyl glycidyl ether at room temperature for double-bond modification of the glutaraldehyde crosslinked porcine pericardium for 72 h, and the solvent of the double-bond solution was 30% (v/v) aqueous ethanol solution.

After the double-bond modification was completed, the double-bond glutaraldehyde crosslinked porcine pericardium was washed with deionized water; and then soaked in a mixture of 20 mM ammonium persulfate and 10 mM sodium bisulfite to further initiate the polymerization reaction of the double bonds on the double-bond glutaraldehyde crosslinked porcine pericardium and react at 37° C. for 7 h, thereby obtaining the porcine pericardium after double-bond polymerization, which was recorded as sample 3 and numbered as GAGA-PP-3.

### Example 4

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an isopropanol aqueous solution of 5% (v/v) glycidyl methacrylate and 2% (v/v) glycidyl acrylate at room temperature for double-bond modification of the glutaraldehyde-crosslinked porcine pericardium for 72 h, and the solvent of the double-bond solution was 35% (v/v) isopropanol aqueous solution.

After the double-bond modification was completed, the double-bond glutaraldehyde crosslinked porcine pericardium was washed with deionized water; and then soaked in a mixture of 20 mM sodium persulfate and 5 mM sodium bisulfite to further initiate the polymerization reaction of the double bonds on the double-bond glutaraldehyde crosslinked porcine pericardium and react at 37° C. for 8 h, thereby obtaining the porcine pericardium after double-bond polymerization, which was recorded as sample 4.

### Example 5

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was washed with deionized water and soaked in an ethanol aqueous solution of 4% (v/v) glycidyl methacrylate at room temperature for double-bond modification for 72 h, and the solvent of the double-bond solution was 20% (v/v) ethanol aqueous solution.

After the double-bond modification was completed, ammonium persulfate and sodium bisulfite were added to initiate the polymerization reaction of the double bonds on the double-bond glutaraldehyde crosslinked porcine pericardium, wherein the concentration of ammonium persulfate was 20 mM and the concentration of sodium bisulfite was 5 mM; after the initiator was added and reacting at 37° C. for 8 h, thereby obtaining the porcine pericardium after double-bond polymerization, which was recorded as sample 5.

### Example 6

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an isobutanol aqueous solution of 4% (v/v) glycidyl methacrylate at room temperature for double-bond modification of the glutaraldehyde crosslinked porcine pericardium for 72 h, and the solvent of the double-bond solution was 15% (v/v) isobutanol aqueous solution.

After the double-bond modification was completed, the double-bond glutaraldehyde crosslinked porcine pericardium was washed with deionized water; and then soaked in a mixture of 20 mM ammonium persulfate and 5 mM sodium bisulfite to further initiate the polymerization reaction of the double bonds on the double-bond glutaraldehyde crosslinked porcine pericardium and react at 37° C. for 8 h, thereby obtaining the porcine pericardium after double-bond polymerization, which was recorded as sample 6.

### Example 7

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an isopropanol aqueous solution of 4% (v/v) glycidyl acrylate at room temperature for double-bond modification of the glutaraldehyde crosslinked porcine pericardium for 48 h, and the solvent of the double-bond solution was 20% (v/v) methanol aqueous solution.

After the double-bond modification was completed, the double-bond glutaraldehyde crosslinked porcine pericardium was washed with deionized water; and then soaked in a mixture of 20 mM ammonium persulfate and 6.5 mM sodium sulfite to further initiate the polymerization reaction of the double bonds on the double-bond glutaraldehyde crosslinked porcine pericardium and react at 37° C. for 10 h, thereby obtaining porcine pericardium after double-bond polymerization, which was recorded as sample 7.

### Example 8

Freshly collected porcine pericardium was washed with distilled water at 4° C. and 100 RPM for 2 h, soaked in 0.30% (w/w) glutaraldehyde solution at room temperature, and soaked for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in an ethylene glycol aqueous solution of 4% (v/v) glycidyl methacrylate at room temperature for double-bond modification of the glutaraldehyde crosslinked porcine pericardium for 72 h, and the solvent of the double-bond solution was 20% (v/v) ethylene glycol aqueous solution.

After the double-bond modification was completed, the double-bond glutaraldehyde crosslinked porcine pericardium was washed with deionized water; and then soaked in a mixture of 40 mM ammonium persulfate and 15 mM sodium bisulfite to further initiate the polymerization reaction of the double bonds on the double-bond glutaraldehyde crosslinked porcine pericardium and react at 37° C. for 7 h, thereby obtaining the porcine pericardium after double-bond polymerization, which was recorded as sample 8.

### Example 9

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was further washed with deionized water and soaked in a propanol aqueous solution of 7% (v/v) glycidyl acrylate at room temperature for double-bond modification of the glutaraldehyde crosslinked porcine pericardium for 60 h, and the solvent of the double-bond solution was 40% (v/v) propanol aqueous solution.

After the double-bond modification was completed, the double-bond glutaraldehyde crosslinked porcine pericardium was washed with deionized water; and then soaked in a mixture of 30 mM sodium persulfate and 10 mM sodium bisulfite to further initiate the polymerization reaction of the double bonds on the double-bond glutaraldehyde crosslinked porcine pericardium and react at 37° C. for 8 h, thereby obtaining the porcine pericardium after double-bond polymerization, which was recorded as sample 9.

### Example 10

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

After washing with deionized water, the glutaraldehyde-crosslinked porcine pericardium was soaked in an isopropanol aqueous solution of 6% (v/v) glycidyl methacrylate and 3% (v/v) glycidyl acrylate at room temperature for double-bond modification of the glutaraldehyde crosslinked porcine pericardium for 84 h, and the solvent of the double-bond solution was 50% (v/v) ethanol aqueous solution.

After the double-bond modification was completed, the double-bond glutaraldehyde crosslinked porcine pericardium was washed with deionized water; and then soaked in a mixture of 40 mM ammonium persulfate and 10 mM sodium sulfite to further initiate the polymerization reaction of the double bonds on the double-bond glutaraldehyde crosslinked porcine pericardium and react at 37° C. for 12 h, thereby obtaining the porcine pericardium after double-bond polymerization, which was recorded as sample 10.

### Performance Characterization of Samples of Examples 1 to 10 and Control Group 1:

In order to characterize the change in the crosslinking degree of glutaraldehyde crosslinked biological valve materials before and after double-bond polymerization and crosslinking treatment, the thermal stability and crosslinking degree of biological valve materials were characterized by measuring the thermal shrinkage temperature of biological valve materials; the stability of biological valve materials was characterized through enzyme degradation experiment; and the degree of calcification (anti-calcification performance) of the samples were characterized through rat subcutaneous implantation experiment.

### Thermal Shrinkage Temperature Determination:

The biological valve material was cut into a circular sheet with a diameter of 0.6 cm, which was placed in a crucible after drying, and then the thermal shrinkage temperature of the biological valve material was measured on a differential scanning calorimeter in the range of 40° C. to 120° C. at a heating rate of 10° C./min. Thermal stability and crosslinking degree of the biological valve material were characterized by measuring the thermal shrinkage temperature. The higher the thermal shrinkage temperature, the higher the thermal stability and crosslinking degree.

TABLE 1
Thermal shrinkage temperature of samples

| | Thermal shrinkage temperature |
|---|---|
| Sample | (° C.) |
| Control group 1 (glutaraldehyde | 84.7 |
| crosslinked porcine pericardium) | |
| Example 1 | 88.9 |
| Example 2 | 89.3 |
| Example 9 | 91.5 |
| Example 10 | 92.0 |

Through the thermal shrinkage temperature measurement of Example 1, Example 2, Example 9, Example 10 and the control group 1 (glutaraldehyde crosslinked porcine pericardium), it can be seen that, as shown in Table 1, the thermal shrinkage temperatures of Example 1, Example 2, Example 9, Example 10 are all higher than that of control group 1 (glutaraldehyde crosslinked porcine pericardium), that is, the thermal stability and crosslinking degree of Example 1, Example 2, Example 9, Example 10 were all higher than those in the control group 1 (glutaraldehyde crosslinked porcine pericardium). The experimental results of the thermal shrinkage temperature measurement show that the method for preparing biological valve materials followed by double-bond polymerization of the present disclosure can improve the thermal stability and crosslinking degree of biological valves.

### Enzyme Degradation Experiment

Sample 3, sample 6, sample 10 and control group 1 were cut into circular sheets with a diameter of 1 cm, with each group consisting of 6 parallel samples. All circular sheet samples were placed on a 48-well plate, frozen overnight at −80° C., and then transferred to a vacuum freeze-dryer to freeze-dry for 48 h. Each sample was weighed on a microbalance as the initial weight (WO) and placed back into the 48-well plate. 0.5 mL collagenase I solution in PBS was added with a pipette to the wells of the 48-well plate so that the biological valve sample is completely immersed in the collagenase solution in PBS (100 U/mL). Then the 48-well plate was transferred to a 37° C. constant temperature incubator for incubation for 24 h. After the incubation, the solution in the well plate was discarded, and a dropper with a rubber bulb was used to draw deionized water to repeatedly flush the biological valve sample in the well plate. After repeated flushing three times, the sample was frozen overnight at −80° C. and then transferred to a vacuum freeze-dryer to freeze-dry for 48 h. Each sample after degradation by collagenase solution was weighed on a microbalance as the final weight (Wt). The calculation formula for enzymatic degradation weight loss rate is as follows:

Enzyme degradation

$$weight loss rate = \frac{{W}_{0} - {W}_{t}}{{W}_{0}} \times 100 \% .$$

The results are shown in Table 2:

TABLE 2
Enzyme degradation weight loss rate of samples

| | Enzyme degradation weight loss rate |
|---|---|
| Sample | (%) |
| Control group 1 (glutaraldehyde | 7.45 ± 1.33 |
| crosslinked porcine pericardium) | |
| Sample 3 | 5.31 ± 0.30 |
| Sample 4 | 4.47 ± 1.05 |
| Sample 6 | 5.12 ± 0.97 |
| Sample 10 | 3.06 ± 0.59 |

The enzyme degradation experiments were conducted on control group 1(glutaraldehyde crosslinked porcine pericardium), sample 3, sample 4, sample 6 and sample 10 to characterize the crosslinking degree of samples, and the enzymatic degradation weight loss rates of samples after control group 1 (glutaraldehyde crosslinked porcine pericardium), sample 3, sample 4, sample 6 and sample 10 were treated with collagenase I were then calculated as shown in Table 2. The enzymatic degradation weight loss rates of sample 3, sample 4, sample 6 and sample 10 were all lower than that of the control group (glutaraldehyde crosslinked porcine pericardium), which indicates that sample 3, sample 4, sample 6 and sample 10 have higher stability than the control group (glutaraldehyde crosslinked porcine pericardium), that is, sample 3, sample 4, sample 6 and sample 10 have higher stability. The results of the enzyme degradation experiment show that the method for preparing biological valve materials followed by double-bond polymerization of the present disclosure can improve the stability of biological valve materials.

### Anti-Calcification Test

The biological valve materials were cut into 0.8*0.8 cm² sheets, sterilized and implanted subcutaneously in rats for 30 days before being taken out. Each sample was divided into two parts. One part was decapsulated and freeze-dried and weighed, and then digested with 6 M hydrochloric acid to measure the calcium content per gram of sample. The other part of the sample was fixed with paraformaldehyde tissue fixative. After fixation, the sampled were taken out and smoothed with a scalpel, and then transferred to a dehydration box. The material samples were dehydrated using gradient ethanol. After dehydration, the material samples were transferred to an embedding machine and embedded in melted wax, and then transferred to a −20° C. refrigerator to cool and trimmed. Slices with 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner

TABLE 3
Calcium content of samples after subcutaneous
implantation in rats for 30 days

| Sample | Calcium content (mg/g) |
|---|---|
| Control group 1 (glutaraldehyde | 74.9 ± 12.3 |
| crosslinked porcine pericardium) | |
| Sample 1 | 15.1 ± 4.7 |
| Sample 5 | 8.4 ± 4.6 |
| Sample 7 | 12.7 ± 5.1 |

The calcium contents of sample 1, sample 5, sample 7 and control group 1 (glutaraldehyde crosslinked porcine pericardium) after being implanted subcutaneously in rats for 30 days was measured to characterize the degree of calcification of samples. As shown in Table 3, the calcium contents of sample 1, sample 5, sample 7 and control group 1 after being implanted subcutaneously in rats for 30 days were all lower than that of the control group (glutaraldehyde crosslinked porcine pericardium). This result indicates that the method for preparing biological valve materials after double-bond crosslinking can improve the anti-calcification performance of biological valves.

### Alizarin Red Staining Experiment

Sample 1, sample 5, sample 7 and control group 1 were implanted into the subcutaneous tissue of rats for 30 days and then taken out and fixed with paraformaldehyde tissue fixative. After fixation, the sampled were taken out and smoothed with a scalpel, and then transferred to a dehydration box. The material samples were dehydrated using gradients of 50%, 75%, 85%, 95% (v/v) and absolute ethanol. After dehydration, the material samples were transferred to an embedding machine and embedded in melted wax, and then transferred to a −20° C. refrigerator to cool and trimmed. Slices with 3 to 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

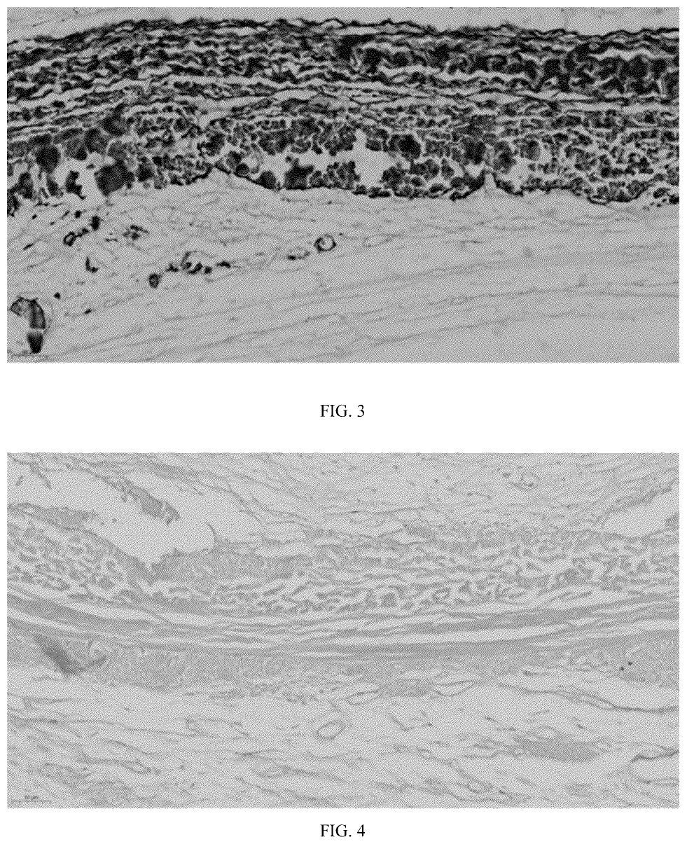

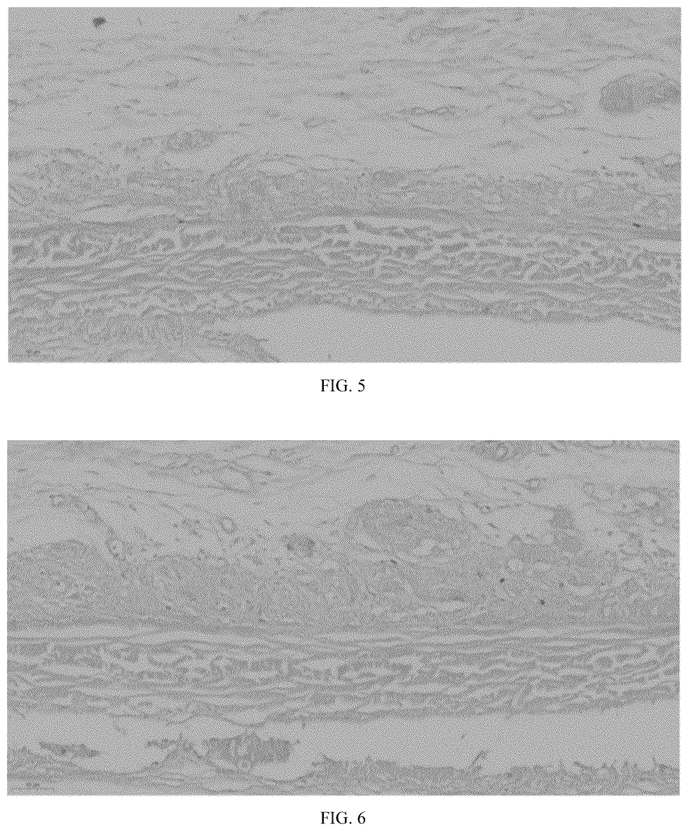

The alizarin red staining experiment was conducted on control group 1 (glutaraldehyde crosslinked porcine pericardium), sample 1, sample 5, and sample 7 after being implanted subcutaneously in rats for 30 days to directly observe the degree of calcification of samples. Images of the alizarin red staining results of sample slices after being implanted subcutaneously in rats for 30 days are shown in FIGS. 3 to 6, wherein the darker the color of the sample after alizarin red staining, the higher the degree of calcification. Compared with the alizarin red staining result of slice of control sample 1 (glutaraldehyde crosslinked porcine pericardium) (FIG. 3), the color of the alizarin red staining results of slices of sample 1 (FIG. 4), sample 5 (FIG. 5), and sample 7 (FIG. 6) is obviously lighter, which directly indicates that the degree of calcification of sample 1, sample 5, and sample 7 is lower than that of the control group 1, that is, compared with control group 1, sample 42, sample 46, and sample 48 have stronger anti-calcification effect. The alizarin red staining results of biological valve materials after being implanted subcutaneously in rats for 30 days show that the method for preparing biological valve materials after double-bond crosslinking of the present disclosure can improve the anti-calcification performance of biological valves.

### Example 11

Freshly collected porcine pericardium was washed with distilled water at 4° C. and 100 RPM for 2 h, soaked in 0.30% (w/w) glutaraldehyde solution at room temperature for 48 h to perform glutaraldehyde crosslinking treatment on the biological valve to obtain glutaraldehyde crosslinked porcine pericardium.

The glutaraldehyde crosslinked porcine pericardium was washed with deionized water and soaked in an ethanol aqueous solution of 4% (v/v) glycidyl methacrylate at room temperature for double-bond modification of the glutaraldehyde crosslinked porcine pericardium for 72 h, and the solvent of the double-bond solution was 20% (v/v) ethanol aqueous solution.

After the double-bond modification was completed, ammonium persulfate and sodium bisulfite were added to initiate the polymerization reaction of the double bonds on the double-bond glutaraldehyde crosslinked porcine pericardium, wherein the concentration of ammonium persulfate was 20 mM and the concentration of sodium bisulfite was 5 mM; after the initiator was added and reacting at 37° C. for 8 h, the porcine pericardium after double-bond crosslinking was obtained.

The porcine pericardium after double-bond polymerization was soaked in a 70% ethanol aqueous solution for 20 minutes, and then soaked in a drying solution (80% glycerol, 2% water, 18% ethanol) for 1.5 h at room temperature. The excess glycerol on the porcine pericardium material was removed, which was sterilized with ethylene oxide, and recorded as sample 31.

### Example 12

Fresh porcine pericardium was placed in PS solution of sodium deoxycholate (surfactant) with a mass fraction of 0.5%, shaken at room temperature for 4 h, and then washed with sodium chloride aqueous solution (i.e. physiological saline) with a mass fraction of 0.9% three times.

The cleaned porcine pericardium was washed in distilled water at 4° C. and 100 RPM for 2 h, and then soaked in 0.30% (w/w) glutaraldehyde solution at room temperature for 48 h for glutaraldehyde crosslinking to obtain glutaraldehyde crosslinked porcine pericardium.

The glutaraldehyde crosslinked porcine pericardium was further washed with deionized water and soaked in a propanol aqueous solution of 5% (v/v) glycidyl methacrylate at room temperature for double-bond modification of the glutaraldehyde crosslinked porcine pericardium for 72 h, and the solvent of the double-bond solution was 20% (v/v) propanol aqueous solution.

After the double-bond modification was completed, the double-bond glutaraldehyde crosslinked porcine pericardium was washed with deionized water; and then soaked in a mixture of 20 mM potassium persulfate and 10 mM sodium bisulfite to further initiate the polymerization reaction of the double bonds on the double-bond glutaraldehyde crosslinked porcine pericardium and react at 37° C. for 8 h, thereby obtaining the porcine pericardium after double-bond crosslinking, which was recorded as sample 34.

The above-described embodiments only illustrate several embodiments of the present disclosure, and the description thereof is specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure. Therefore, the protection scope of the present disclosure should be determined by the appended claims.

What is claimed is:

1. A method for preparing a biological valve material by means of double-bond polymerization after aldehyde crosslinking, comprising:
Step S110: contacting biomaterial with an aldehyde-based crosslinking agent solution for crosslinking;
Step S120: soaking the biomaterial treated in step S110 in a solution containing a first functional monomer for a chemical reaction to introduce first carbon-carbon double bonds;
wherein the first functional monomer has the first carbon-carbon double bonds and an ethylene oxide group; and
Step S200: performing polymerization of the carbon-carbon double bonds under an action of an initiator to obtain the biological valve material.

2. The method according to claim 1, wherein the aldehyde-based crosslinking agent is glutaraldehyde or formaldehyde.

3. The method according to claim 1, wherein the biomaterial is an animal tissue selected from one or more of the following: pericardium, valve, intestinal valve, meninges, lung valve, blood vessel, skin or ligament.

4. The method according to claim 3, wherein the animal tissue is a fresh animal tissue or a decellularized biological tissue.

5. The method according to claim 1, wherein the step S200 comprises:
adding the initiator to a system treated in a previous step; or
taking the biomaterial treated in the previous step out and directly or after washing soaking the biomaterial treated in the previous step in a solution containing the initiator.

6. The method according to claim 1, wherein the initiator is a single initiator or a mixed initiator.

7. The method according to claim 6, wherein the mixed initiator is:
a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; and a concentration of each component in the mixture is in a range of 1 to 100 mM.

8. The method according to claim 7, wherein the single initiator is any component of the mixed initiator.

9. The method according to claim 1, wherein in step S200, a double-bond polymerization time is in a range of 3 to 24 h.

10. The method according to claim 1, wherein the first functional monomer is at least one selected from a group consisting of allyl glycidyl ether, glycidyl methacrylate and glycidyl acrylate.

11. The method according to claim 1, wherein in step S110:
a w/w concentration of the aldehyde-based crosslinking agent solution is in a range of 0.1% to 5%; and a crosslinking time is in a range of 0.5 h to 120 h.

12. The method according to claim 1, wherein in step S120:
a w/w concentration of the first functional monomer in the solution containing the first functional monomer is in a range of 1% to 10%; and a reaction time is in a range of 2 to 120 h.

13. The method according to claim 1, wherein the solution containing the first functional monomer only contains the first functional monomer and a solvent that does not participate in chemical reaction.

14. The method according to claim 1, wherein the solvent in the solution containing the first functional monomer is one or more of the following: water, physiological saline, a neutral pH buffer, and an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol.

15. A biological valve material prepared by the method according to claim 1.

16. A biological valve material prepared by the method according to claim 5.

17. A biological valve material prepared by the method according to claim 10.

18. A biological valve, comprising a stent and leaflets, wherein the leaflets are made of the biological valve material according to claim 15.

19. The biological valve according to claim 18, wherein the biological valve is a prosthetic heart valve.

20. An interventional system, comprising a prosthetic heart valve and a catheter assembly. wherein the prosthetic heart valve is delivered by the catheter assembly after being folded. wherein the prosthetic heart valve comprises a stent and leaflets, and the leaflets are made of the biological valve material according to claim 15.
